SEQUENCE LISTING (5 sequences)

SEQ ID NO: 1 - protein, 16 aa, human
HLIHNVHLEEHAHAHN

SEQ ID NO: 2 - protein, 18 aa, human
HDDHDDHDDHDDHDDHDD

SEQ ID NO: 3 - protein, 18 aa, human
HEEHEEHEEHEEHEEHEE

SEQ ID NO: 4 - protein, 18 aa, human
HDEHDEHDEHDEHDEHDE

SEQ ID NO: 5 - protein, 18 aa, human
HEDHEDHEDHEDHEDHED

(19) United States
(12) Patent Application Publication
(10) Pub. No.: US 2009/0084166 A1
(43) Pub. Date: Apr. 2, 2009

(54) Sensors comprising a chelated metal ion linker layer

(76) Inventor: Karla M. Robotti, Cupertino, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/904,686

(22) Filed: Sep. 27, 2007

(51) Int. Cl. G01N 33/487; B01J 19/00; B05D 1/36

(52) U.S. Cl. 73/53.01; 422/68.1; 427/402; 427/407.1

(57) ABSTRACT

Embodiments of sensors according to the invention include a substrate having an activated dielectric surface; a metal ion chelate layer on the activated dielectric surface; and a bifunctional layer on a surface of said metal ion chelate layer, where the bifunctional layer includes bifunctional molecules that have a metal ion affinity moiety; and a ligand attachment moiety. Ligands are located at one or more positions on the surface of the bifunctional layer in certain embodiments. Aspects of the invention further include methods of making the sensors, as well as kits for practicing the methods. Further aspects of the invention include methods of using the sensors, e.g., in analyte detection applications.

### INTRODUCTION

Sensitive and accurate methods for detecting molecular interactions are very desirable for a wide variety of applications. Such applications include drug discovery, environmental testing, diagnostics, gene expression analysis, genomics analysis, and proteomics, as well as in applications designed to characterize the binding of two molecules that are known to bind together.

One technique that is employed for detecting molecular interactions is surface plasmon resonance (SPR). Reviews of SPR may be found in, e.g., Homola, J., et al., Sensors and Actuators B 54: 3-15 (1999); Welford, K., Opt. Quant. Elect. 23:1 (1991); Raether, H., Physics of Thin Films 9: 145 (1977). In a SPR assays for detecting molecular interactions, a ligand that selectively binds to an analyte of interest is bound to the metal surface. The interaction of the sensing surface with an analyte in a solution in contact with the surface is then monitored.

In such SPR-based assays, the ligand is bound to the metal surface of the sensor in a manner such that binding of analyte to the ligand can be detected. Binding of ligands like proteins and nucleic acids to metal surfaces can be challenging. As such, a variety of different ligand attachment techniques have been developed. However, the development of additional ligand attachment techniques that provide for sensors having low non-specific binding during use is desirable.

### SUMMARY

Sensors in accordance with the invention include a substrate having an activated dielectric surface; a metal ion chelate layer on the activated dielectric surface; and a bifunctional layer on a surface of the metal ion chelate layer. The bifunctional layer includes bifunctional molecules that have a metal ion affinity moiety and a ligand attachment moiety. In certain embodiments, ligands are located at one or more positions on the surface of the bifunctional layer. Aspects of the invention further include methods of making the sensors, as well kits for practicing the methods. Further aspects of the invention include methods of using the sensors, e.g., in analyte detection applications.

### BRIEF DESCRIPTION OF THE FIGURES

FIG. 1A provides a side view showing an example of a sensor according to an embodiment of the invention.

FIG. 1B provides a side view showing the sensor shown in FIG. 1A, where ligands are linked to the surface of the sensor. The ligands are linked to the surface by a chelated metal ion linking layer.

FIG. 2 provides a side view showing a system that includes the sensor shown in FIG. 1B.

### DEFINITIONS

The term “analyte” is used herein to refers to a known or unknown component of a sample. Analytes specifically bind to a ligand on a substrate surface if the analyte and the ligand are members of a specific binding pair. Analytes are chemical molecules of interest. Analytes may be polymers, e.g., biopolymers, where biopolymers of interest include, but are not limited to, nucleic acids, including oligonucleotides and polynucleotides, peptides, polypeptides and antibodies. An “analyte” is referenced herein as a moiety in a mobile phase (such as a fluid). The analyte is one that is to be detected by a “ligand” which is bound to a substrate. In applications described herein, either of the “analyte” or “ligand” may be the entity which is to be evaluated by the other (thus, either one could be an unknown mixture of analytes, e.g., polypeptides, to be evaluated by binding with the other).

An “array” includes any one, two-dimensional or substantially two-dimensional (as well as a three-dimensional) arrangement of addressable regions (i.e., locations) bearing a particular chemical moiety or moieties (e.g., biopolymers such as polynucleotide or oligonucleotide sequences (nucleic acids), polypeptides (e.g., proteins), carbohydrates, lipids, etc.) associated with that region. In an array, the combination of chemical moiety or moieties at a specific location is referred to as a feature. Arrays of interest include arrays of bio polymeric binding agents. An array may contain more than ten, more than one hundred, more than five hundred features, in an area of less than 20 cm² or even less. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature).

The term “assessing” includes any form of measurement, and includes determining if an element is present or not. The terms “determining”, “measuring”, “evaluating”, “assessing” and “assaying” may include quantitative and/or qualitative determinations. Assessing may be relative or absolute. “Assessing the presence of” includes determining the amount of something present and/or determining whether it is present or absent.

If one composition is “bound” to another composition, the compositions do not have to be in direct contact with each other. In other words, bonding may be direct or indirect. If two compositions (e.g., a substrate and a ligand) are bound to each other, there may be at least one other composition (e.g., another layer) between those compositions. Binding between any two compositions described herein may be covalent or non-covalent. In the context of chemical structures, “bound” (or “bonded”) may refer to the existence of a chemical bond directly joining two moieties or indirectly joining two moieties (e.g., via a linking group). The chemical bond may be a covalent bond, an ionic bond, a coordination complex, hydrogen bonding, van der Waals interactions, or hydrophobic stacking, or may exhibit characteristics of multiple types of chemical bonds.

A “biopolymer” is a polymer of one or more types of repeating units, regardless of the source. The biopolymer may be naturally-occurring, e.g., where it is obtained from a cell-based recombinant expression system. Biopolymers may also be synthetic. Biopolymers of interest include, but are not limited to, nucleic acids, including oligonucleotides and polynucleotides, peptides, polypeptides and antibodies. Biopolymers may be of any length, e.g., 2 monomers or greater, 4 monomers or greater, 10 monomers or greater, 20 monomers or greater, 50 monomers or greater, 100 monomers or greater, 300 monomers or greater, up to 500, 1000 or 10,000 or more monomers in length.

The term “ligand” refers to an agent that binds an analyte through a specific binding interaction. Ligands of interest include any moiety that can specifically bind to an analyte. In certain embodiments, a polypeptide (e.g., a monoclonal antibody or a peptide), a nucleic acid (e.g., DNA or RNA), a polysaccharide, or other biopolymer may be employed.

The term “ligand/analyte complex” is a complex that results from the specific binding of a ligand with an analyte. As used herein, “binding partners” and equivalents refer to pairs of molecules that can be found in a ligand/analyte complex, i.e., exhibit specific binding with each other. A ligand and an analyte for the ligand will specifically bind to each other under “conditions suitable for specific binding” (also referenced as “specific binding conditions”), where such conditions are those conditions (in terms of salt concentration, pH, detergent, protein concentration, temperature, etc.) which allow for binding to occur between ligands and analytes in solution. Such conditions, particularly with respect to proteins and nucleic acids are include but are not limited to those described in Harlow and Lane (Antibodies: A Laboratory Manual Cold Spring Harbor Laboratory, Cold Spring Harbor, N.Y. (1989) and Ausubel, et al., Short Protocols in Molecular Biology, 5th ed., Wiley & Sons, 2002). “Hybridizing” refers to specific binding of nucleic acids.

“Optional” or “optionally” means that the subsequently described circumstance may or may not occur, so that the description includes instances where the circumstance occurs and instances where it does not.

The term “polypeptide” refers to compounds that have at least two covalently attached amino acids. Proteins, peptides and oligopeptides are all types of polypeptides. A polypeptide may be made up of naturally occurring amino acids and peptide bonds, synthetic peptidomimetic structures, or a mixture thereof. Thus “amino acid”, or “peptide residue”, as used herein encompasses both naturally occurring and synthetic amino acids. “Amino acid” also includes imino acid residues such as proline and hydroxyproline. The side chains may be in either the D- or the L-configuration. The term “polypeptide” includes polypeptides in which the conventional backbone has been replaced with non-naturally occurring or synthetic backbones, The term also includes polypeptides in which one or more of the conventional amino acids have been replaced with one or more non-naturally occurring or synthetic amino acids. The term “fusion protein” or grammatical equivalents thereof references a protein composed of a plurality of polypeptide components. In fusion proteins, polypeptide components that are not attached in their native state are joined by their respective amino and carboxyl termini through a peptide linkage to form a single continuous polypeptide.

The term “pre-determined” refers to an element whose identity is known prior to its use. For example, a “pre-determined analyte” is an analyte whose identity is known prior to any binding to a ligand. An element may be known by name, sequence, molecular weight, its function, or any other attribute or identifier.

A “prism” is a structure that is bounded in part by two nonparallel plane faces and is used to refract or disperse a beam of light. A prism comprises a light-transmissive material.

The term “sample” as used herein relates to a material or mixture of materials. The sample may be in fluid form. The sample may contain one or more components of interest present in fluid medium, such as an aqueous or non-aqueous solvent. Samples may be derived from a variety of sources such as from food stuffs, environmental materials, a biological sample such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components).

The term “specific binding” refers to the ability of a ligand to preferentially bind to a particular analyte that is present in a homogeneous mixture of different analytes. The binding interaction is mediated by an affinity region of the ligand for the analyte of interest. A specific binding interaction will discriminate between desirable and undesirable analytes in a sample, such as by 10 to 100-fold or more (e.g., 1000- or 10,000-fold or more). The affinity between a ligand and analyte when they are specifically bound in a ligand/analyte complex is characterized by a K_{D} (dissociation constant) of 10⁻⁴ M or less, 10⁻⁵ M, 10⁻⁶ M, 10⁻⁷ M, 10⁻⁸ M, 10⁻⁹ M, or 10⁻¹⁰ M, or even less. In contrast to specific binding interactions, non-specific binding interactions are interactions between to entities that are not mediated by an affinity region of one entity for the other.

The phrase “surface-bound ligand” refers to a ligand that is immobilized on a surface of a solid s. Such “surface bound ligands” may be bound directly to the substrate or indirectly bound to the substrate, e.g., via one or more intermediate moieties and/or layers of intermediate materials. In certain embodiments, the ligands employed herein are present on a surface of the same substrate.

### DETAILED DESCRIPTION

Sensors in accordance with the invention include a substrate having an activated dielectric surface; a metal ion chelate layer on the activated dielectric surface; and a bifunctional layer on a surface of said metal ion chelate layer. The bifunctional layer includes bifunctional molecules that have a metal ion affinity moiety and a ligand attachment moiety. During use, ligands may be located at one or more positions on the surface of the bifunctional layer. Aspects of the invention further include methods of making the sensors, as well kits for practicing the methods. Further aspects of the invention include methods of using the sensors, e.g., in analyte detection applications.

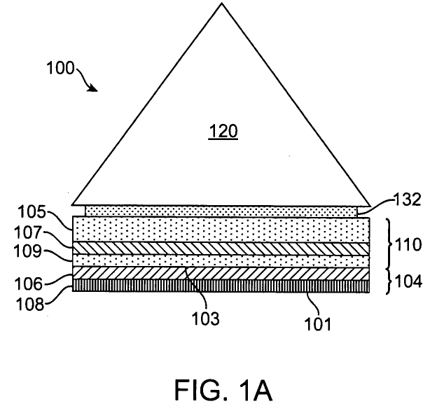

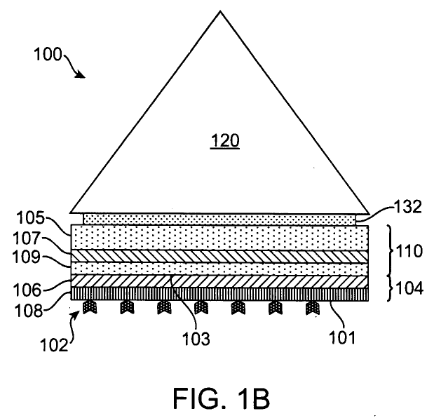

With reference to FIG. 1A, a sensor 100 includes a substrate 110 having an activated dielectric layer. The surface 103 of the substrate has been functionalized in some manner such that it is capable of bonding to a chelated metal ion linking layer (described in greater detail below). Functional groups that may be present on surface 103 include, but are not limited to: hydroxyl groups, carboxylic acids and aldehydes. When the chelated metal ion linking layer is present on the surface 103, the chelated metal ion linking layer is immobilized on the activated dielectric surface of the substrate 110. In certain embodiments, the chelated metal ion linking layer is covalently bonded to the surface 103 of the substrate The substrate having an activated dielectric layer 110 includes a light-transmissive support 105, a metal layer 107 on a surface of the light-transmissive support and a dielectric (e.g., glass) layer 109 on a surface of the metal layer opposite the metal layer 107. Chelated metal ion linker layer 104 is also shown. Chelated metal ion linker layer 104 includes a bifunctional layer 108 that is bound to the surface 103 via a metal ion chelate layer 106.

In the embodiment shown in FIG. 1A, sensor 100 includes a prism 120 in operable relation to the light-transmissive support 105. As shown, the light-transmissive support 105 is placed adjacent an edge of the prism 120 to receive light transmitted through the prism 120. A refractive index-matching composition 132 (e.g., an oil, gel, adhesive) may be included between the light-transmissive support 105 and prism 120 in such configurations. In certain embodiments, the light-transmissive support 105 is prism-shaped, in which case no separate prism needed. In certain embodiments, the sensor does not include a prism but is disposed with light-transmissive support 105 adjacent a surface of a prism.

The light-transmissive support 105 may have various shapes and/or sizes adapted to the intended use in the sensor substrate. In certain embodiments, the light-transmissive-support 105 has at least one planar surface. Examples of configuration for the light-transmissive support 105 include slides, sheets, pads, slices, films, strips, disks, etc.

The light-transmissive support 105 comprises one or more materials which permit the transmission of light through the material. Materials employed for the light-transmissive support 105 include, but are not limited to, one or more transparent materials selected from glass, quartz, silica, a polymeric material, such as an acrylic polymer, cyclic olefin, polyolefin, polydimethylsiloxane, polymethylmethyl acrylate, and/or a polycarbonate.

Positioned on a surface of the light-transmissive support 105 is metal layer 107. The layer of metal 107 may be a free electron metal such as, e.g., copper, silver, aluminum or gold, although other metals may be used, such as a metal selected from platinum, palladium, chromium, niobium, rhodium, and iridium. Different metals produce different resonance effects, and, as such, the choice of metal depends on the resonance effect desired. This metal layer 107 may be positioned on a surface of the light-transmissive support using any convenient protocol, e.g., sputtering, coating or evaporation. The metal layer 107 generally has a uniform thickness. In certain embodiments, the thickness of the metal layer 107 ranges from 20 nm to 120 nm, such as from 20 nm to 60 nm. A metal grating, may also be present in a subject sensor. In certain embodiments, more than one metal is present in the metal layer, e.g., the metal layer may include a first layer of one metal and another layer of a second metal.

Positioned on a surface of the metal layer 107 opposite the light-transmissive support 105 is dielectric layer 109. This layer 109 generally has a uniform thickness. In certain embodiments, layer 109 is 50 rum or thinner, e.g., 40 nm, 30 nm, 20 nm, 10 nm thick or thinner, and is, in certain embodiments, 2 or more nm thick. This dielectric layer 109 is distinct from the light-transmissive support 105. The dielectric layer 109 is separated from the light-transmissive support 105 by metal layer 107. The dielectric layer is, in certain embodiments, a layer of silicon dioxide. In certain instances, the silicon dioxide layer may be deposited directly on the metal layer. In yet other instances, an adhesion promoting layer, e.g., a layer of silicon nitride, may be positioned between the silicon dioxide layer and the metal surface.

In producing the sensor, the layers of materials bound to the light-transmissive support 105 that make up the activated dielectric surface of the substrate may be produced using any convenient fabrication protocol. Fabrication protocols employed will depend, at least in part, on the nature of the particular layer to be produced. Protocols that may be employed in fabrication of the various layers include, but are not limited to sputtering, evaporation, chemical vapor deposition, and plasma-enhanced chemical vapor deposition.

As indicated above, the surface 103 of dielectric layer 109 is activated, such that it displays functional groups, such as hydroxyl functional groups. Depending on the protocol employed to deposit the dielectric layer 109, the dielectric layer may initially display the desired functional groups without further modification. In yet other embodiments, an activating process may be employed to produce the desired functional groups on the surface of the dielectric layer. For example, where silicon dioxide is the dielectric layer, an activating process may be employed that disrupts surface Si—O bonds on the surface of the layer to provide —OH functional groups. Activating treatments of interest include, but are not limited to: plasma treatment, e.g., exposure to oxygen plasma, exposure to strong acids, acids having a pH of 2 or less e.g., nitric acid, etc.

Present on the activated dielectric surface 110 is a chelated metal ion linking layer 104. The chelated metal ion linking layer is a layer that displays functional groups on its surface for ligand binding and also is immobilized relative to the underlying activated dielectric layer on which it is positioned. The chelated metal ion linking layer further exhibits low non-specific binding, e.g., 400 RU or less, such as 100 RU or less, including 10 RU or less, as determined using the commercially available surface Plasmon resonance instrumentation available from Biacore Life Sciences, a division of GE Healthcare, according to manufacturer's instructions. (RU stands for resonance unit and is directly proportional to the concentration of biomolecules on a surface. 1 RU is ca. 1 pg/mm².)

The chelated metal ion linking layer 104 in generally a layer of uniform thickness. The uniform thickness of the layer may vary so long as surface binding events are detectable by the sensor. In certain embodiments, the chelated metal ion linker layer 104 ranges from 0.5 nm to 200 nm, such as from 1 nm to 100 nm, including from 2 nm to 20 nm. The chelated metal ion linker layer includes a chelated metal ion that is complexed with a metal ion affinity moiety, as reviewed in greater detail below.

Embodiments of the chelated metal ion linker layer include two distinct sub-layers, i.e., a metal ion chelate layer 106 and a bifunctional layer 108. The thickness of the metal ion chelate layer 106 is generally uniform. A given metal ion chelate layer 106 may have a uniform thickness ranging from 1 nm to 100 nm, such as 2 nm to 20 nm and including 5 nm to 10 nm. The metal ion chelate layer 106 includes a first surface and a second opposing surface. The first surface is in contact with the surface of the activated dielectric layer 110, such that the metal ion chelate layer 106 is stably associated with the activated dielectric layer. The second opposing surface displays chelated metal ions. Embodiments of the metal ion chelate layer include layers that comprise metal ions complexed with chelating moieties of polyalkylene glycol molecules. In these embodiments, the metal ion chelate layer 106 is a metal ion complexed polyalkylene glycol layer.

For metal ion chelate layer 106, various polyalkylene glycol molecules may be employed, so long as they include metal ion chelator moieties that are capable of complexing with metal ions. Polyalkylene glycol molecules of interest include, but are not limited to: polyethylene glycol, poly propylene glycol, polybutylene glycol, polyisopropylene glycol, and the like. Polyoxyalkylene glycols used in the invention may have a number average molecular weight of from 100 to 100,000; such as from 1,000 to 50,000; including from 10,000 to 40,000. In certain embodiments, the polyalkylene glycol is polyethylene glycol (i.e., PEG). In certain embodiments, the polyalkylene glycol is a multi-arm polyalkylene glycol, where the multi-arm polyalkylene glycol includes a core structure with two or more polyalkylene arms bonded thereto, e.g., 3 to 20, such as 3 to 12 arms, such as is found in the multi-arm PEG molecules disclosed in U.S. Pat. No. 6,828,401.

As mentioned above, the polyalkylene molecules of the metal ion chelate layer 106 include metal ion chelating functionalities (i.e., moieties). These functionalities can be bonded to the polyalkylene glycol molecules via any convenient linking protocol. Metal ion chelating functionalities of interest include, but are not limited to: iminodiacetic acid (IDA), nitriloacetic acid (NTA), carboxymethylated aspartic acid (CM-Asp), and tris-carboxymethyl ethylene diamine (TED). These functionalities offer a maximum of tri-(IDA), tetra-(NTA, CM-Asp), and penta-dentate (TED) complexes with the respective metal ions.

In the metal ion chelating layers 106, metal ions are complexed with the chelating functionalities. In this manner, the metal ion chelate layer 106 is “loaded” with metal ions. Metal ions of interest include, but are not limited to: hard metal ions, such as Fe³⁺, Ca²⁺, and Al³⁺; soft metal ions, such as Cu⁺, Hg²⁺ and Ag⁺; and intermediate metal ions, such as Cu²⁺, Ni²⁺, Zn²⁺, and Co²⁺

In certain embodiments, the metal ion chelate layer is a chelated Cu²⁺/PEG layer as sold commercially by Microsurfaces Inc. (Minneapolis, Minn.).

The bifunctional layer 108 of the chelated metal ion linker layer 104 is made up of bifunctional molecules having metal ion affinity moieties and ligand attachment moieties. The metal ion affinity moieties are complexed with the metal ions of the metal ion chelate layer 106. As the metal ion affinity moieties of the bifunctional layer 108 are complexed with the metal ions of the metal ion chelate layer 106, the bifunctional layer 108 is stably associated with the metal ion chelate layer 106.

Metal ion affinity moieties of interest include metal ion affinity peptides. Metal ion affinity peptides of interest may range from 6 to 30, from 7 to 25, from 8 to 20, from 9 to 18, from 10 to 16, or from 12 to 14 amino acids in length. In some embodiments, metal ion affinity peptides contain from 30% to 50%, from 33% to 45%, from 35% to 43%, or from 37% to 40%, histidine residues. In some embodiments, metal ion affinity peptides bind to intermediate metal ions with an affinity of from 10³ M⁻¹ to 10⁹ M⁻¹; and to hard metal ions with an affinity of from 10³ M⁻¹ to 10⁹ M⁻¹.

In some embodiments, a metal ion affinity peptide includes 2-6 adjacent histidine residues, e.g., a metal ion affinity peptide includes (His)ₙ, where n=2, 3, 4, 5, or 6. In some embodiments, a metal ion affinity peptide has the formula R₁-(His)ₙ-R₂, where R₁ is hydrogen, or from 1 to 30 amino acids, where n=2-6, and where R₂ is Q, Q-Ile-Glu-Gly-Arg- or Q-Asp-Asp-Asp-Asp-Lys-, where Q is a peptide bond or from 1 to 30 amino acids. See, e.g., U.S. Pat. No. 5,310,663.

In other embodiments, a metal ion affinity peptide has the formula His-X, where X is chosen from -Gly-His, -Tyr, -Gly, -Trp, -Val, -Leu, -Ser, -Lys, -Phe, -Met, -Ala, -Glu, -Ile, -Thr, -Asp, -Asn, -Gln, -Arg, -Cys, and -Pro. In some of these embodiments, the metal ion affinity peptide is chosen from His-Trp, His-Tyr, His-Gly-His, and His-Phe. In other embodiments, a metal ion affinity peptide has the formula Y-His, where Y is chosen from Gly-, Ala-, and Tyr-. See e.g., U.S. Pat. No. 4,569,794.

In some embodiments, a metal ion affinity peptide has the formula R₁-(His-X)ₙ where R₁ is a hydrogen atom, an amino acid, or a sequence of from two amino acids to 10 amino acids, or a sequence of from 10 amino acids to 50 amino acids, or a polypeptide; where X is selected from Asp, Pro, Glu, Ala, Gly, Val, Ser, Leu, Ile, and Thr or a combination of any of the foregoing amino acids; and where n=3-6. See, e.g., U.S. Pat. No. 5,594,115.

In some embodiments, a metal ion affinity peptide comprises a peptide of the formula: (His-(X₁)ₙ)ₘ, wherein m≧3, wherein X₁ is any amino acid other than His, wherein n=1-3, provided that, in at least one His-(X₁)ₙ unit, n>1. In some embodiments, a metal ion affinity peptide comprises a peptide of the formula: (His-X₁-X₂)ₙ₁-(His-X₃-X₄-X₅)ₙ₂-(His-X₆)ₙ₃ wherein each of X₁ and X₂ is independently an amino acid with an aliphatic or an amide side chain, each of X₃, X₄, X₅ is independently an amino acid with a basic side chain (except His) or an acidic side chain, each X₆ is an amino acid with an aliphatic or an amide side chain, n1 and n2 are each independently 1-3, and n3 is 1-5. In some embodiments, each of X₁ and X₂ is independently selected from the group consisting of Leu, Ile, Val, Ala, Gly, Asn, and Gln. In other embodiments, each of X₁ and X₂ is independently selected from the group consisting of Leu, Val, Asn, and Ile. In some embodiments, each of X₃, X₄, Xs is independently selected from the group consisting of Lys, Arg, Asp, and Glu. In some embodiments, each of X₃, X₄, XS is independently selected from the group consisting of Lys and Glu. In some embodiments, each X₆ is independently selected from the group consisting of Leu, Ile, Val, Ala, Gly, Asn, and Gln. In other embodiments, each X₆ is independently selected from the group consisting of Ala and Asn. In one particular embodiment, the metal ion affinity peptide has the amino acid sequence NH₂-His-Leu-Ile-His-Asn-Val-His-Lys-Glu-Glu-His-Ala-His-Ala-His-Asn-COOH (SEQ ID NO:01).

In certain embodiments, the metal ion affinity peptide has the formula (His-Asn)ₙ, wherein n=3 to 10. In certain embodiments, n=from 4 to 10, such as from 5 to 10. In one particular embodiment, n=6. In certain embodiments, the affinity peptide has the formula (His-X₁-X₂)ₙ, wherein each of X₁ and X₂ is an amino acid having an acidic side chain, and n=3 to 10. In one embodiment, the affinity peptide comprises the sequence (His-Asp-Asp)₆ (SEQ ID NO:02). In another embodiment, the affinity peptide comprises the sequence (His-Glu-Glu)₆ (SEQ ID NO:03). In a further embodiment, the affinity peptide comprises the sequence (His-Asp-Glu)₆ (SEQ ID NO:04). In a further embodiment, the affinity peptide comprises the sequence (His-Glu-Asp)₆ (SEQ ID NO:05).

In certain embodiments, the bifunctional molecule is a polypeptide. The polypeptide includes a metal ion affinity peptide at a first end and a ligand attachment moiety at a second end. The overall length of the polypeptide may vary, ranging from 5 to 100 residues in length, such as 5 to 50 residues in length, including 5 to 25 residues in length, e.g., 10 to 15 residues in length. The non-metal ion affinity residues may, in certain embodiments, be any convenient residues, where residues of interest include, but are not limited to: Gly, Ala, Ile, Leu and the like.

The surface 101 of the bifunctional layer 108 displays ligand attachment moieties. The ligand attachment moiety is any moiety that participates in binding a ligand to the bifunctional layer 108, e.g., by reacting chemically with the ligand to form a covalent bond. With respect to the ligand attachment moiety, any convenient coupling chemistry compatible with the sensor substrate (i.e., which does not result in degradation of the sensor substrate) may be used to couple to the ligand. Strategies of interest employ complementary reactive groups on the ligand or are selected based on moieties already present on the ligand (e.g., amino groups of peptides). Ligand attachment moieties of interest include, but are not limited to: carboxy, amino, etc.

FIG. 1B provides a side view of sensor 100 having ligands 102 bound to bifunctional layer 108. Ligands 102 may be any moiety that specifically binds an analyte through an interaction that is sufficient to permit the ligand to bind and concentrate the analyte from a homogeneous mixture of different analytes. The ligand may be selected based on its ability to bind to the desired analyte, either directly or indirectly. A directly binding ligand is a single molecule that binds to an analyte of interest. An indirectly binding ligand is a complex of two or more molecules that operate together to specifically bind an analyte of interest. Examples of indirectly binding ligands are ligands that include an avidin/biotin interaction.

The ligand may be a moiety capable of binding to one or more of food stuffs, environmental materials, a biological sample such as tissue or fluid isolated from an individual (including but not limited to, for example, plasma, serum, spinal fluid, semen, lymph fluid, the external sections of the skin, respiratory, intestinal, and genitourinary tracts, tears, saliva, milk, blood cells, tumors, organs), and also samples of in vitro cell culture constituents (including but not limited to conditioned medium resulting from the growth of cells in cell culture medium, putatively virally infected cells, recombinant cells, cell components, and cell fragments). In certain embodiments, the ligand may be a moiety isolated from food stuffs, environmental materials, a biological sample such as tissue or fluid isolated from an individual (including but not limited to, for example, plasma, serum, spinal fluid, semen, lymph fluid, the external sections of the skin, respiratory, intestinal, and genitourinary tracts, tears, saliva, milk, blood cells, tumors, organs), and also samples of in vitro cell culture constituents (including but not limited to conditioned medium resulting from the growth of cells in cell culture medium, putatively virally infected cells, recombinant cells, cell components, and cell fragments).

In certain embodiments, the ligand is a biopolymer. In certain embodiments, the ligand is a polypeptide, e.g., an antibody, a peptide, a protein, an enzyme, a fragment thereof. In certain other embodiments, the ligand may be a polynucleotide (i.e., nucleic acid), such as a RNA fragment, a DNA fragment, an oligonucleotide, or a synthetic mimetic of a polynucleotide (e.g., a peptidonucleic acid “PNA” or other modified nucleic acids). In some embodiments, the ligand may be a cell, a cell fragment, a bacterium, a spore, a virus, or a virion. In some embodiments, the ligand may be a drug compound or an organic compound known to specifically bind to an analyte.

Ligands finding use in embodiments of the invention can be pre-made (e.g., isolated from a source, synthesized by a machine, or made by recombinant means) and then be bound to the sensor. Alternatively, ligands may be synthesized in situ on the sensor substrate.

In some embodiments, ligands 102 can be attached to the linker layer through additional chemical layers, e.g., layers that provide for increased ligand density on the surface of the sensor. These additional chemical layers have either a 2-dimensional or 3-dimensional quality. The 2-dimensional layer may be a self-assembled monolayer formed atop the linker layer. In such embodiments, all ligands would attach at the terminal end of the monolayer, thus positioning the ligands at a uniform distance from the chelated metal ion linker layer. The 3-dimensional layer would be formed when the linker layer is further attached to a layer that includes multiple ligand attachment sites, e.g., a hydrogel layer or a dendrimer-type layer. In such embodiments, the attachment sites are distributed throughout the gel layer.

In certain embodiments, different ligands may be present on a sensor surface. In this context, the term “different” denotes a different molecular formula such that the ligands are different molecules of different molecular formula). In these embodiments, the sensor surface 101 has a two or more sites distinct sites. Each distinct site (i.e., feature) has a different ligand bound thereto via layer 104. In such embodiments, the sensor includes an array of different ligands addressably located on surface 101.

Aspects of the invention also include detection systems. Detection systems in accordance with the invention include sensors that include a chelated metal ion linking layer, e.g., as described above. In certain embodiments, the detection systems are evanescent wave sensor systems. Evanescent wave sensor systems include, but are not limited to systems based on: surface plasmon resonance, grating coupler surface plasmon resonance, resonance mirror sensing and waveguide sensor interferometry using Mach-Zender or polarimetric methods, direct and indirect evanescent wave detection methods, etc., as described in, e.g., Homola, J., et al., Sensors and Actuators B 54: 3-15 (1999); Welford, K., Opt. Quant. Elect. 23:1 (1991); Raether, H., Physics of Thin Films 9: 145 (1977); Myszka, J. Mol. Rec. 12:390-408 (1999); and Biomolecular Sensors, edited by Gizeli and Lowe. Taylor & Francis (2002).

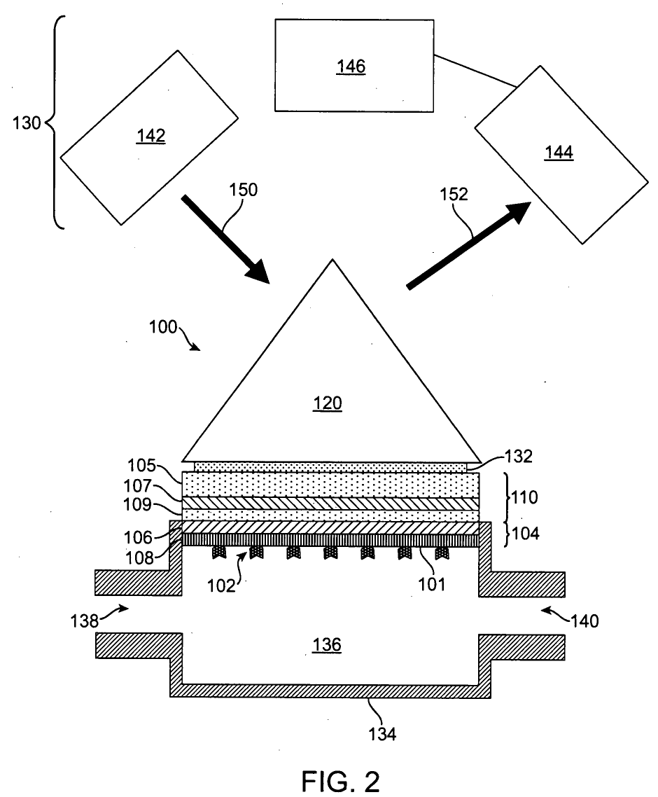

Referring to the embodiment illustrated in FIG. 2, the system 200 contains a sensor 100 in accordance with the invention disposed in operable relation to an optical detection system 130. A housing 134 is coupled to sensor 100 and defines a fluid flow chamber 136 with sensor 100. During use, ligands 102 are exposed to sample when sample is flowed through chamber 136. Sample is introduced into chamber 136 via fluid inlet 138 and exits chamber 136 via fluid outlet 140. Fluid inlet 138 and fluid outlet 140 are in fluid communication with appropriate fluid feeds and valves to control the flow of liquid sample into and out of the chamber 136. Depending on the design of the analyte detection system, housing 134 may be an integral part of the sensor. Alternatively, housing 134 may be attached to the sensor, allowing for replacement of the sensor and housing as a unit.

Optical detection system 130 includes light source 142 and optical detector 144. Optical detector 144 is connected to detection circuitry 146. Detection circuitry 146 may be in the form of a processor programmed with suitable software. Detection circuitry 146 may be, but is not necessarily, a computer-based system. Light source 142 may be a wavelength-tunable laser or other light source. In certain embodiments, light source 142 provides light having a wavelength of between 400 nm to 2.0 μm when used in the subject methods. In certain embodiments, the wavelength of light used is from 0.6 to 1.2 μm, e.g., 0.7 μm to 1.0 μm. In certain embodiments, the wavelength of light used may change between two or more different wavelengths during a given reading of a sensor.

During use, light source 142 directs a light beam 150 toward prism 120 by way of suitable optics. Light beam 150 passes into and through light-transmissive support 105 and reflects off metal layer 107. Reflected light 152 is received by optical detector 144. Optical detector 144 passes a corresponding signal to the detection circuitry 146. The ligand 102 is contacted with sample and may bind to analytes in the sample. Binding of analyte and ligand 102 causes a change in a property of the reflected light 152 that can be detected by optical detection system 130. Upon detection of the change in a property of the reflected light 152, the detection system may produce a sample-responsive signal. The resultant sample responsive signal may then be employed to assess the presence of analyte in the sample. In certain embodiments, the sample-responsive signal is in the form of a “sensorgram.” A sensorgram is a plot of response (measured in “resonance units” or “RU”) as a function of time. An increase of RU corresponds to an increase of mass on the sensor surface. As sample containing an analyte contacts the sensor surface, the ligand bound to the sensor surface interacts with the analyte in a step referred to as “association.” This step is indicated on the sensorgram by an increase in RU as the sample is initially brought into contact with the sensor surface. Conversely, “dissociation” normally occurs when sample flow is replaced by, for example, a buffer flow. This step is indicted on the sensorgram by a drop in RU over time as analyte dissociates from the surface-bound ligand. In this manner, the analyte detection system uses SPR to detect binding of an analyte.

In certain embodiments, the methods further include contacting the sensor with a known ligand (i.e., a sample having a known concentration of a known ligand) and obtaining a known ligand-responsive signal from the sensor. This known ligand-responsive signal may be employed in such methods as a reference value for use in evaluated a sample-responsive signal. In certain embodiments, the sensor is contacted with the known ligand before the sensor is contacted with the sample to obtain a ligand-responsive signal. In yet other embodiments, the sensor is contacted with the known ligand after the sensor is contacted with the sample to obtain a ligand-responsive signal. In using the known ligand-responsive signal as a reference, the method further includes comparing a sample-responsive signal to the ligand-responsive signal to produce a result.

Where the sensor includes an array of ligands, e.g., as described above, the system can be configured to simultaneously detect binding at different locations of the array. For example, optical detector 144 can include a pixellated image sensor (not shown), such as a complimentary metal oxide semiconductor (CMOS) image sensor or a charge coupled device (CCD) and suitable optics (not shown) that form an image the array surface of the sensor on the image sensor. The image sensor is employed to generate corresponding signals for different locations of the array. In this manner, data for an entire sensor or for selected sections of a sensor can be collected simultaneously.

Results from reading a subject sensor may be raw results or may be processed results. Processed results include those obtained by applying saturation factors to the readings, rejecting a reading which is above or below a predetermined threshold and/or any conclusions from the results (such as whether or not a particular analytes may have been present in the sample). The results of the reading (processed or not) may be forwarded (such as by communication) to a remote location if desired, and received there for further use (such as further processing). Stated otherwise, in certain variations, the subject methods may include transmitting data to the remote location for further evaluation and/or use. Any convenient telecommunications means may be employed for transmitting the data, e.g., modem, internet, wireless protocols, etc. Alternatively, or in addition, the data representing results may be stored on a computer-readable medium of any variety.

Specific analyte detection applications of interest include hybridization assays in which the nucleic acid ligands are employed and protein binding assays in which polypeptide ligands, e.g., antibodies or peptides, are employed. Additional applications of interest include those described in U.S. Pat. Nos. 7,262,866; 7,012,694; 6,844,201; 6,808,938; 6,775,003; 6,589,798; 6,503,760; 6,289,286; 6,207,381; 6,143,574; 6,143,513; 6,127,183; 5,972,612; 5,965,456; 5,955,729 and 5,641,640; the disclosures of which methods and systems are herein incorporated by reference.

Aspects of the invention also include kits. Kits include a sensor in accordance with the invention, e.g., as described above. Such a kit may also include instructions and/or reagents for customizing the sensor to display one or more ligands of interest. In certain embodiments, the sensor provided in the kit may already display one or more ligands. In certain embodiments, kits may also include reagents for preparing samples and/or refractive index-matching compositions. The kits may also include one or more control analyte mixtures, e.g., two or more control analytes for use in testing the kit. The various components of the kits may be present in separate containers. Certain compatible components may be pre-combined into a single container, as desired.

In addition to above-mentioned components, kits in accordance with the invention may further include instructions for using the components of the kit to practice the subject methods. The instructions are recorded on a suitable recording medium. For example, the instructions may be printed on a medium, such as paper or plastic, etc. As such, the instructions may be present in the kits as a package insert, in the labeling of the container of the kit or components thereof (e.g., associated with the packaging or subpackaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable medium included in the kit.

The following examples are offered by way of illustration and not by way of limitation.

### EXPERIMENTAL

### A. Production of Substrate Having an Activated Dielectric Surface 110

Gold-coated glass slides (titanium adhesion layer of 5 nm and evaporated gold of 46.5 nm) are further treated with an evaporated layer of 310 nm of silicon nitride followed with 30 nm of silicon dioxide via plasma-enhanced chemical vapor deposition (PECVD). This dielectric coated slide was submitted to MicroSurfaces, Inc. (Minneapolis, Minn.) for a commercially-available deposition of a copper-chelated PEG layer.

### B. Preparation of Chelated Metal Ion Linker Layer 104 for Ligand Attachment

Slides having a metal ion linker layer 106 were coated with a 10% glycerol solution containing 0.1% of 6×His-tetraglycine for 30 minutes to produce bifunctional layer 108 bound to the surface of the metal ion linker layer 108. Slides were washed with 1×-phosphate-buffered saline, water and dried. This step results in the production of chelated metal ion linker layer 104.

### C. Ligand Attachment

The sensor produced in B above is placed in a system as shown in FIG. 2 so that surface 101 is exposed to chamber 136. Ligands can be attached to the slide surface 101 with a 7 min. injection of a 1:1 ratio of 0.4 M EDC(N-ethyl-N′-dimethylaminopropylcarbodiimide) and 0.1 M NHS(N-hydroxysuccinimide) through chamber 136. The ligand, diluted in an appropriate buffer, is then coupled to this area with a 7 min. injection and a flow of 5 μl/min through chamber 136. Finally, a 7 min injection of 1.0 M ethanolamine is flowed over area to deactivate any remaining attachment groups on the surface. When the ligand was the protein, carbonic anhydrase II (CAII), a surface saturation results in ca. 1500 RU of CAII immobilized to the surface. A reference area on the slide is also activated (EDC/NHS) and blocked in the same manner, without any added protein.

### D. Non-Specific Binding Test

To test for non-specific binding, a ligand (CAII) was flowed over surface 101 through chamber 136 in the same manner as above but without any prior activation with EDC/NHS. This resulted in ca. 10 RU of binding (negligible).

### E. Use of Sensor in Biomolecular Interaction Assay

An assay is performed to evaluate the binding of enzyme carbonic anhydrase II (CAII) with the drug, carboxybenzenesulfonamide (CBS).

The ligand, (CAII) is immobilized on the surface layer as stated above in “ligand attachment”. The small molecule analyte, CBS, is first diluted to 1 mg/mL in phosphate-buffered saline. Further dilutions are made to generate a series of concentrations ranging from 40 μM to 80 nM. These drug concentrations are run over the surface area that has CAII attached. Response units are proportional to the concentration of the analyte and are cleanly seen above the baseline. Binding curves are produced of the analyte interaction with the enzyme and binding rates are calculated.

It is to be understood that this invention is not limited to particular embodiments described herein. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

As used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Each of the individual embodiments described and illustrated herein has discrete components and features which may be readily separated from or combined with the features of any of the other several embodiments without departing from the scope of the present invention. Any recited method can be carried out in the order of events recited or in any other order which is logically possible.

Although the foregoing invention has been described in some detail by way of illustration and example for purposes of clarity of understanding, certain changes and modifications may be made thereto without departing from the scope of the appended claims.

I claim:

1. An sensor comprising:
a substrate having an activated dielectric surface;
a metal ion chelate layer on said activated dielectric surface; and
a bifunctional layer on a surface of said metal ion chelate layer, said bifunctional layer comprising bifunctional molecules comprising:
a metal ion affinity moiety; and
a ligand attachment moiety.

2. The sensor according to claim 1, wherein said metal ion chelate layer comprises a metal ion complexed polyalkylene glycol layer.

3. The sensor according to claim 2, wherein said metal ion complexed polyalkylene glycol layer comprises complexed copper ions.

4. The sensor according to claim 2, wherein said metal ion complexed polyalkylene glycol layer is a metal ion complexed polyethylene glycol layer.

5. The sensor according to claim 1, wherein said metal ion affinity moiety is a metal ion affinity peptide.

6. The sensor according to claim 5, wherein said metal ion affinity peptide is poly-histidine peptide.

7. The sensor according to claim 1, wherein said ligand attachment moiety is a carboxyl or amino group.

8. The sensor according to claim 1, wherein said sensor further comprises a ligand bonded to said ligand attachment moiety.

9. The sensor according to claim 1, wherein said substrate comprises:
a light-transmissive support;
a metallic layer on a surface of said light-transmissive support; and
a dielectric layer on a surface of said metallic layer.

10. The sensor according to claim 1, wherein said sensor is configured for use in an evanescent wave sensor system.

11. A method comprising:
a) providing a sensor comprising:
a substrate having an activated dielectric surface;
a metal ion chelate layer on said activated dielectric surface; and
a bifunctional layer on a surface of said metal ion chelate layer,
said bifunctional layer comprising bifunctional molecules comprising:
a metal ion affinity moiety; and
a ligand attachment moiety;
b) contacting said sensor with a sample; and
c) obtaining a sample-responsive signal from said sensor.

12. The method according to claim 11, wherein said contacting comprises flowing said sample across said bifunctional layer.

13. The method according to claim 11, wherein said method further comprises contacting said sensor with a known ligand and obtaining a known ligand-responsive signal from said sensor.

14. The method according to claim 13, wherein said method further comprises comparing said sample-responsive signal to said ligand-responsive signal to produce a result.

15. The method according to claim 14, wherein said method further comprises using said result to determine the presence of an analyte in said sample.

16. The method according to claim 11, wherein said method further comprises introducing said sensor into a reader.

17. The method according to claim 11, wherein said substrate comprises:
a light-transmissive support;
a metallic layer on a surface of said light-transmissive support; and
a dielectric layer on a surface of said metallic layer.

18. A method of making a sensor, said method comprising:
a) providing a substrate having an activated dielectric surface;
(b) producing a metal ion chelate layer on a surface of said activated dielectric surface; and
(c) producing a bifunctional layer on a surface of said metal ion chelate layer, said bifunctional layer comprising bifunctional molecules comprising:
a metal ion affinity moiety; and
a ligand attachment moiety.

19. The method according to claim 18, wherein said metal ion chelate layer is produced by bonding a polyalkylene glycol layer to said surface of said activated dielectric surface, wherein said polyalkylene glycol layer comprises metal ion chelating moieties, and complexing metal ions to said metal ion chelating moieties.

20. The method according to claim 18, wherein said substrate comprises:
a light-transmissive support;
a metallic layer on a surface of said light-transmissive support; and
a dielectric layer on a surface of said metallic layer

21. A kit comprising:
(a) a sensor comprising:
a substrate having an activated dielectric surface;
a metal ion chelate layer on said activated dielectric surface; and
a bifunctional layer on a surface of said metal ion chelate layer, said bifunctional layer comprising bifunctional molecules comprising:
a metal ion affinity moiety; and
a ligand attachment moiety; and
(b) a reagent for use with said sensor.

22. The kit according to claim 21, wherein said reagent is a ligand or a buffer.

23. The kit according to claim 21, wherein said substrate comprises:
a light-transmissive support;
a metallic layer on a surface of said light-transmissive support; and
a dielectric layer on a surface of said metallic layer.
